(19) United States
(12) Patent Application Publication
(10) Pub. No.: US 2004/0068769 A1
(43) Pub. Date: Apr. 8, 2004

(54) Enhancement of freezing tolerance in transgenic plants

(76) Inventors: Fathey Sarhan, Saint-Laurent (CA); Mario Houde, Longueuil (CA); Sylvain Dallaire, Montreal (CA); Daniel N'Dong, Laval (CA)

Correspondence Address: CHRISTINA HILDEBRAND, NORRIS, MCLAUGHLIN & MARCUS, 220 EAST 42ND STREET - 30TH FLOOR, NEW YORK, NY, 10017, US

(21) Appl. No.: 10/415,134

(22) PCT Filed: Oct. 29, 2001
(86) PCT No.: PCT/CA01/01499

(30) Foreign Application Priority Data
Oct. 27, 2000 (CA) 2,321,944

(51) Int. Cl. A01H 1/00; C12N 15/82; C07K 14/415

(52) U.S. Cl. 800/289; 530/370

(57) ABSTRACT

The invention relates to a method of increasing the viability of a plant cell comprising transforming a plant with a nucleic acid encoding a cold tolerance polypeptide from the Wcor410 family and combining this treatment with a period of low temperature exposure.

### FIELD OF THE INVENTION

The present invention relates to engineering cells, tissues and organs, from animals or plants for low temperature, dehydration or salt tolerance by using a cold tolerance gene from cold-tolerant plants.

The invention also relates to the method used to efficiently transform and generate stable transgenic strawberry plants. This method was efficient in producing a cold-tolerant strawberry using a gene from a cold tolerant species. The gene encodes a protein involved in protecting the cellular membrane against osmotic stress caused by freezing, salt, and dehydration. The gene product could be useful in protecting leaves flowers and fruit against these osmotic stresses. This will result in extending shelf life of these fresh produce.

The invention also teach the use of cold tolerance genes or their products to protect different molecules, cells, tissues and organs during storage at low temperature.

### BACKGROUND OF THE INVENTION

Cold acclimation (CA) allows hardy plants to develop efficient tolerance mechanisms needed for winter survival. During this period, numerous biochemical, physiological, and metabolic functions are altered in plants. These changes are regulated by low temperature (LT) at the gene expression level. To understand the molecular basis of adaptation to LT, efforts were focussed on identifying LT-responsive genes. Cold-regulated genes and their products have been isolated and characterized in numerous species¹⁻³. In wheat and other cereals, the level of expression of several genes, such as wcs120, wcs19 and wcor410, during CA is a heritable trait that correlates with the capacity of each genotype to develop freezing tolerance (FT)⁴⁻⁶. Among these, the wcor410^{7,8} gene family encodes highly abundant membrane-associated proteins. This family belongs to a subfamily of dehydrin, proteins that are associated with the dehydration stress caused by freezing. Although their precise function is still unknown, their high hydrophilicity, stability at high temperature and their association with the plasma membrane suggest that they are involved in the protection of the plasma membrane against freezing stress. The plasma membrane is a very important site for water exchange during dehydration and lipid demixing can occur during severe dehydration⁹. Thus, the accumulation of the WCOR410 proteins may play an important role in maintaining plasma membrane integrity and could be a determining factor for increased cell resistance to freezing. To evaluate the impact of this protein on the membrane stability and freezing tolerance in a commercial crop, we transferred the wheat Wcor410a cDNA into an elite strawberry cultivar (Fragaria×anassassa, cv Chambly)¹² using an improved transformation procedure to obtain pure and stable transformed lines of strawberry. there are several differents genes known to be induced by exposure to low temperature or for which expression is correlated with freezing tolerance. However, we could not predict which ones will have a real impact on improving low-temperature of freezing temperature tolerance.

### SUMMARY OF THE INVENTION

The invention relates to a method of increasing the viability of a plant cell comprising transforming a plant with a nucleic acid encoding a cold tolerance polypeptide from the Wcor410 family and combining this treatment with a period of low temperature exposure.

In another embodiment, the invention includes a method of improving low temperature tolerance comprising transforming a plant with a nucleic acid encoding a cold tolerance polypeptide from the Wcor410 family and combining this treatment with a period of low temperature exposure.

The invention also includes a method of improving freezing tolerance of a plant comprising transforming a plant with a nucleic acid encoding a cold tolerance polypeptide from the Wcor410 family and combining this treatment with a period of low temperature exposure.

Another variation of the invention includes a method of improving cross-stress tolerance comprising transforming a plant with a nucleic acid encoding a cold tolerance polypeptide from the Wcor410 family and combining this treatment with a period of low temperature exposure.

The invention also includes a method of improving membrane stability; increasing the viability of a plant cell; improving low temperature tolerance or improving freezing tolerance of a plant; comprising transforming a plant with a nucleic acid encoding a cold tolerance polypeptide from the Wcor410 family.

The invention also includes a method of increasing the viability of a plant cell; improving low temperature tolerance; improving freezing tolerance of a plant or improving cross-stress tolerance in a plant comprising transforming a plant with a nucleic acid encoding a cold tolerance polypeptide from the Wcor410 family and combining this treatment with a period of low temperature exposure.

Another embodiment of the invention includes a method of improving fruit and leafy vegetable stability at low temperature; improving seed stability and shelf life at low temperature or prolonging shelf life of fruit and leafy vegetable at low temperature comprising transforming a plant with a nucleic acid encoding a cold tolerance polypeptide from the Wcor410 family.

Another aspect of the invention relates to a method of producing pure stable transgenic strawberry lines using high selection pressure, preferably high kanamycin selection pressure. In one embodiment, the invention includes a method of transforming a plant with a nucleic acid comprising contacting the plant with an Agrobacterium tumefaciens suspension in the presence of at least 450 mg/L of kanamycin.

The invention also includes a method of improving the metabolic stability and activity of a cells stored at low temperature or cryoprotection of a cell for biomedical applications comprising transforming the cell with a nucleic acid encoding a cold tolerance polypeptide from the Wcor410 family.

Another aspect of the invention includes a method of improving plant productivity; improving plant resistance to osmotic stress; improving plant resistance to pathogen stress comprising transforming a plant with a nucleic acid encoding a cold tolerance polypeptide from the Wcor410 family.

The invention also includes a method of improving and increasing the shelf life and quality of frozen food or increasing the shelf life of a drug stored at low temperature comprising adding to the food an effective amount of a cold tolerance polypeptide from the Wcor410 family.

In any of the above methods the wcor410 family preferably comprises Wcor410a, Wcor410b and Wcor410c. The nucleic acid preferably comprises the wcor410 nucleic acid in FIG. 1 (SEQ ID NO:1) or nucleotide nos. 87 to 875 of (SEQ ID NO:1). The nucleic acid may also comprise a nucleic acid having at least 50% sequence identity to (SEQ ID NO:1). The nucleic acid may also be a nucleic acid that hybridizes to (SEQ ID NO:1) under stringent hybridization conditions comprising 55-65 degrees Celsius, 5×SSC, 2% SDS; wash: 60-65 degrees Celsius, 0.1×SSC, 0.1% SDS. In any of the above methods, the polypeptide preferably comprises the WCOR410 amino acid sequence in FIG. 1 (SEQ ID NO:2). The polypeptide also usefully comprises a polypeptide having at least 50% sequence identity to (SEQ ID NO:2).

The invention also includes a host cell comprising a nucleic acid encoding a cold tolerance polypeptide from the Wcor410 family, or progeny of the host cell. The host cell is preferably selected from the group consisting of a fungal cell, a yeast cell, a bacterial cell, a microorganism cell and a plant cell. The invention also includes a plant, a plant part, a seed, a plant cell or progeny thereof comprising a nucleic acid encoding a cold tolerance polypeptide from the Wcor410 family. The plant part may be all or part of a leaf, a flower, a stem, a root or a tuber. The plant, plant part, seed or plant cell may be a dicot plant or a monocot plant. The Wcor410 family sequences described in this application (as well as sequences having identity or that hyrbridize, as described below) may be inserted in the above host cell, plant, plant part, seed or plant cell.

This patent related to a demonstration of the feasibility of improving FT with the WCOR410 gene family.

### BRIEF DESCRIPTION OF THE DRAWINGS

Preferred embodiments of the invention are shown in the drawings:

FIG. 1: nucleic acid (SEQ ID NO.1) and polypeptide (SEQ ID NO:2), preferably wcor410a;

FIG. 2: nucleic acid (SEQ ID NO.3) and polypeptide (SEQ ID NQ:4), preferably wcor410b; and

FIG. 3: nucleic acid (SEQ ID NO.5) and polypeptide (SEQ ID NO:6), preferably wcor410c;

### DETAILED DESCRIPTION OF THE INVENTION

“Low Temperature (LT)” is defined, as temperatures higher than about 0° C. but not lower than a plant can withstand.

“Freezing tolerance (FT)” is defined as normally below 0° C. but not lower than a plant can withstand.

“Growth characteristics” refers to leaf area and/or biomass.

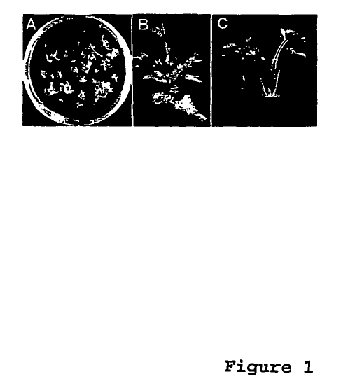

Efficient production of transgenic strawberry was obtained using thidiazuron at 2 mg/L to induce abundant organogenesis during infection with Agrobacterium. After the second subculture of the transformed explants, the concentration of thidiazuron was reduced to 1 mg/L to stimulate growth and differentiation (FIG. 1). The first series of transgenic strawberry was produced using the kanamycin selection pressure recommended for strawberry¹⁰. However, we found that the lines obtained using 200 mg/L kanamycin were unstable and produced mosaic plants where new runners did not express the WCOR410a protein. To increase the genetic stability of transgenic plants, the selection pressure was rapidly increased to 450 mg/L kanamycin. This high concentration of kanamycin had no deleterious effects on strawberry and produced healthy plants that expressed high levels of the transgene (FIG. 1). Three homogeneous transgenic strawberry lines that expressed the WCOR410a protein to a level equivalent to cold-acclimated wheat were selected. These lines were stable through several generations of new plants derived from runners.

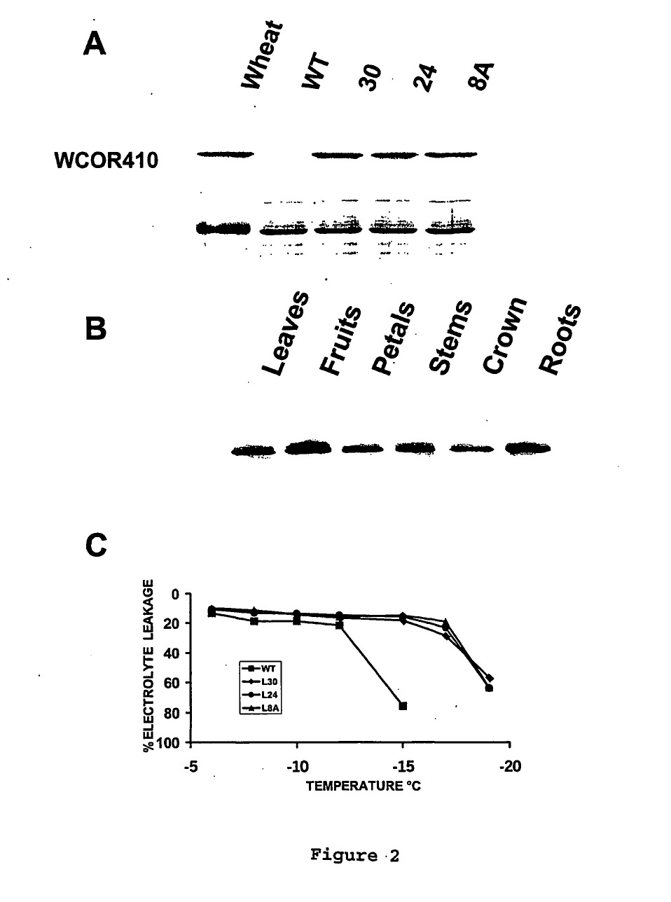

The level of WCOR410 protein expression was measured by immunoblot in the three transgenic lines and in the wild type strawberry before, during, and after cold acclimation. The data in FIG. 2A clearly show that no proteins homologous to WCOR410 were detected in wild type strawberry plants (FIG. 2A). On the other hand, the transgenic lines express the wheat protein at a level, comparable to tolerant winter wheat (FIG. 2A). The WCOR410a protein was found ubiquitously expressed in all tissues indicating that the 35S promoter allowed a uniform expression of this gene in the transgenic strawberry (FIG. 2B).

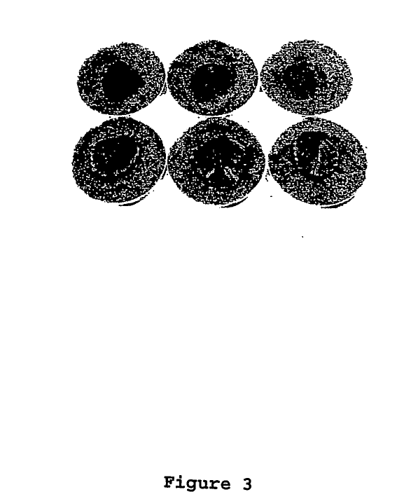

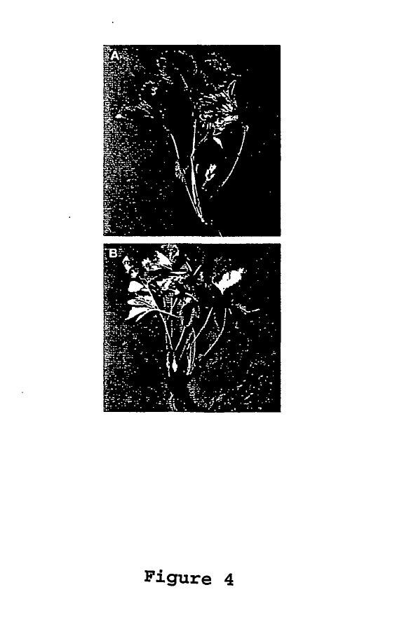

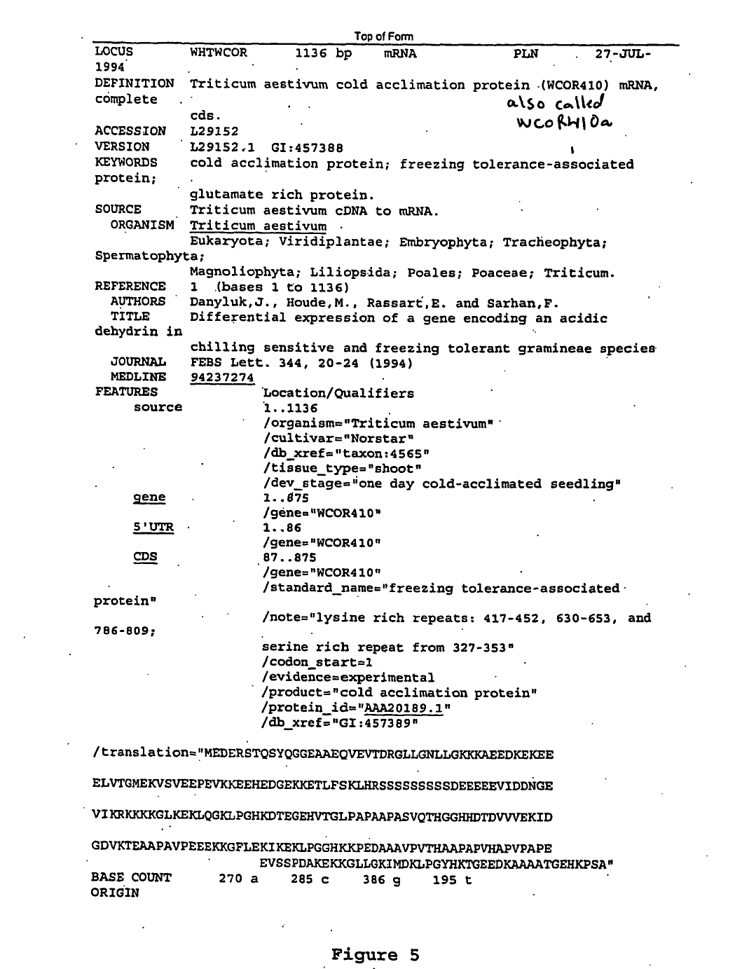

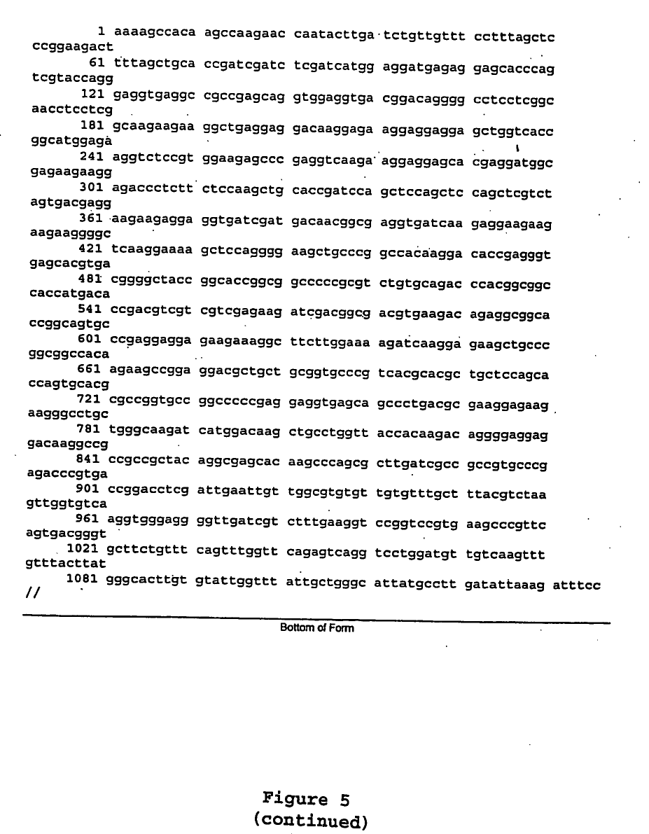

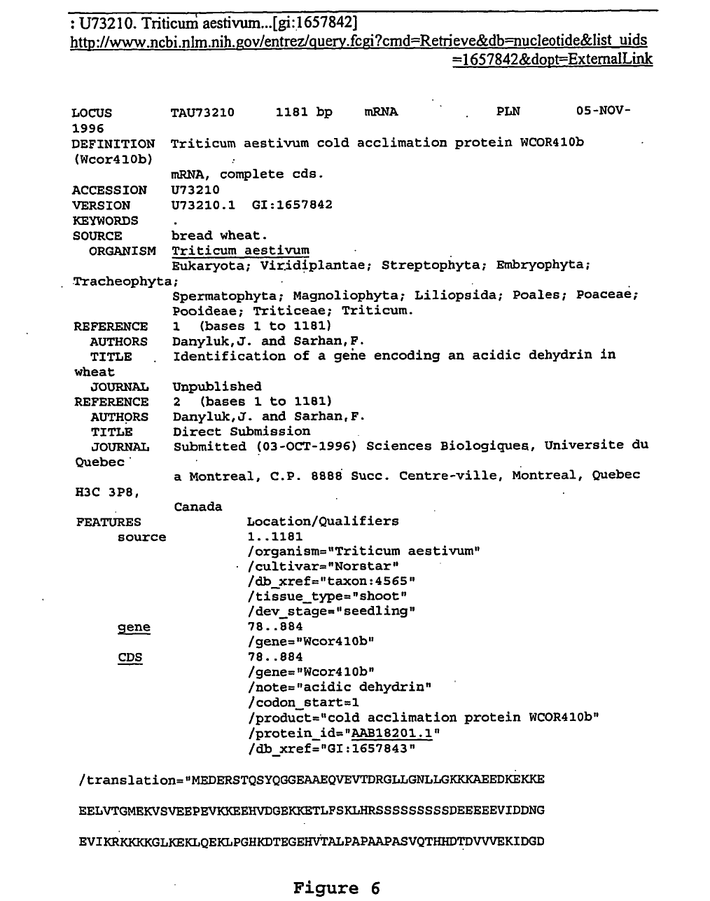

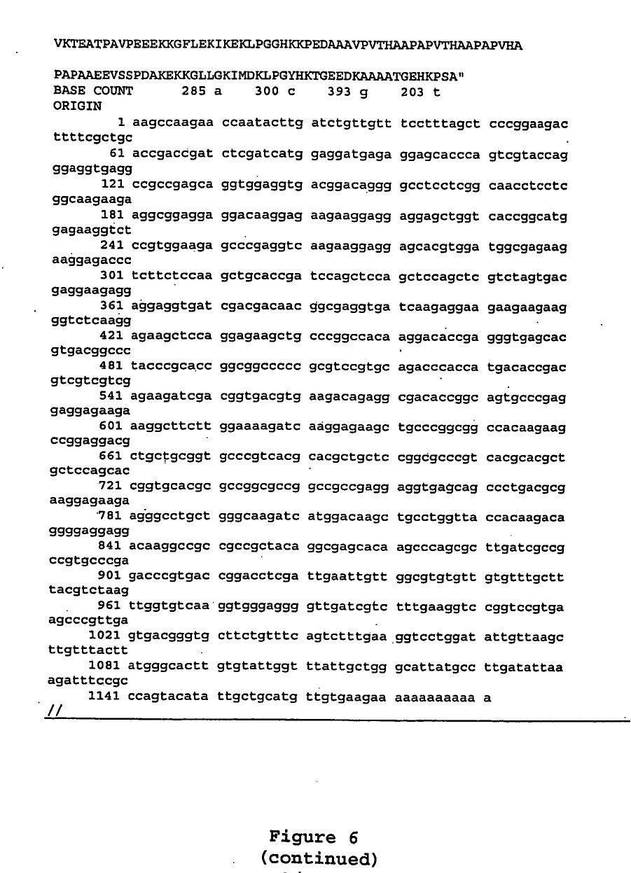

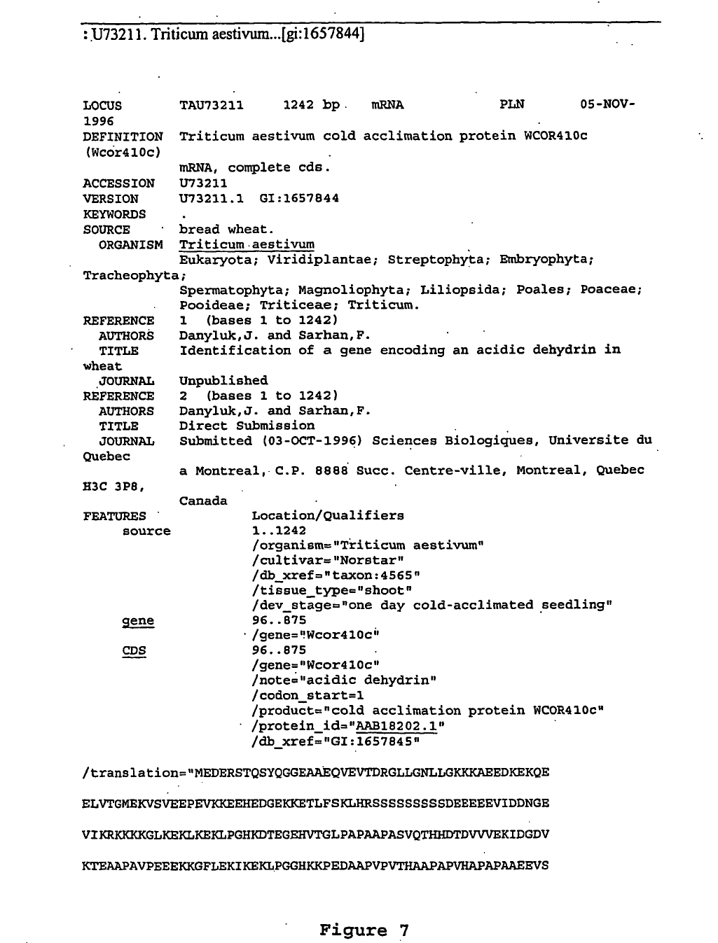

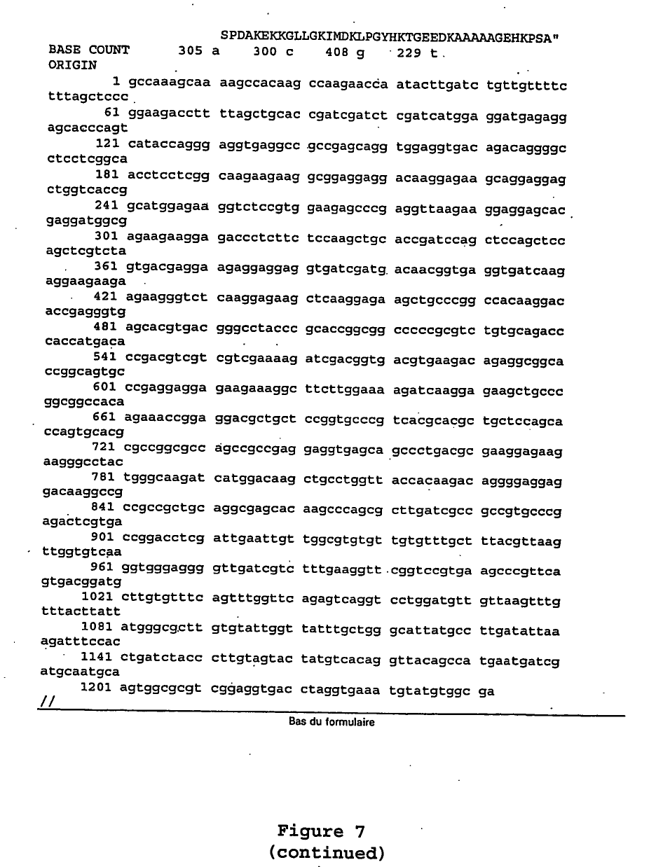

The growth characteristics and freezing tolerance of the wild type strawberry cultivar Chambly was compared with the transgenic lines. No difference in growth behaviour was observed between the transgenic and the wild type cultivar indicating that constitutive over-expression of WCOR410a does not cause any deleterious effect on plant growth. Freezing tests of the transgenic plants grown under non-acclimated conditions (20° C.) showed a slight improvement of FT compared to wild type plants. However, when plants were cold-acclimated, the transgenic plants had greater FT, compared to wild type. An optimal cold acclimation period of three weeks was found to result in the maximal improvement in FT in the transgenic strawberry. These results clearly indicate that the WCOR410a protein, which is constitutively expressed, needs to interact with other factors induced by low temperature exposure to achieve maximal FT. To accurately measure the degree of FT, the LT₅₀ (lethal temperature that kills 50% of the cells) was determined in the wild type and the three transgenic lines after three weeks of cold acclimation using the electrolyte leakage method. This freezing test is a direct measurement of ions released from the cells due to the rupture of the plasma membrane⁸. The data in FIG. 2C shows that 3 weeks cold-acclimated leaves from both the wild type and transgenic strawberry lines were not affected by temperatures down to −12° C. The wild type plants, however, were sensitive to further decrease in temperature since more than 70% of ions leached out of the cells at −15° C. On the other hand, the three transgenic lines did not show any signs of damages at −15° C. and were minimally affected at −17° C. However, a significant ion leakage was observed when the temperature was lowered to −19° C. The LT₅₀ of leaves from the wild type cultivar is estimated at −13° C. while for the three transformed lines, the LT₅₀ of leaves is estimated at −18° C. (FIG. 2C). The increase of 5° C. in FT represents a spectacular improvement. This result is illustrated in FIG. 3 where we can clearly see that leaves from the three transgenic lines remain healthy and very green after freezing at −15° C. while the wild type leaves are dark indicating cell rupture and rapid degradation of chlorophyll due to cell death. The same results were obtained with whole plants (FIG. 4). A similar difference was also observed at −18° C. A similar improvement in FT tolerance was also obtained in Arabidopsis transformed with Wcor410. The transgenic plants were also found to have a higher resistance to other osmotic stresses such as dehydration and salt. These improvements will lead to healthier plants that could also resist other biotic and abiotic and thus increase overall productivity.

We conclude from these experiments that the wcor410 genes (including Wcor410a SEQ ID NO: 1, Wcor410b SEQ ID NO: 3, and Wcor410c SEQ ID NO:5⁸) from wheat plays an important role in protecting plants from freezing injury and thus confers a greater cold tolerance to the plant.

The high expression of the WCOR410a protein in petals also have an important role in protecting flowers during springtime when plants are vulnerable to low temperature damage. The expression in the berries and leaves enhances membrane stability and preserve food nutrient quality during storage at low temperature. Increasing fruit and leafy vegetables shelf life will have an important economic impact for the producers and will increase the market value of the product. The mechanism by which the WCOR410 proteins protects plants cellular membranes allows its efficient use as a cryoprotectant for frozen food and sensitive biological material such as molecules, blood, cells, tissues and organs³. It is useful in developing the cryopreservation technologies applicable to various plants and mammalian cells, tissues, organs or whole organisms including seeds. These proteins have numerous agriculture and bio-medical applications. For example, wheat FT proteins such as WCOR410, WCS120 and WCS19, could be used to improve the viability, plating efficiency, and hepatospecific functions of cryopreserved hepatocytes. They are also useful as agents to preserve the stability and shelf-life of drugs stored at low temperature.

It will be apparent that Wcor410a may be varied, for example, by shortening the 5′ untranslated region or shortening the nucleic acid molecule so that the 3′ end nucleotide is in a different position.

The discussion of the nucleic acid molecules, sequence identity, hybridization and other aspects of nucleic acid molecules included within the scope of the invention is intended to be applicable to either the entire nucleic acid molecule in the figure or its coding region. One may use the entire molecule or only the coding region. Other possible modifications to the sequence are apparent.

Sequence Identity

Nucleic acid sequences having sequence identity to Wcor410a sequence are found in other plants. Derivatives of Wcor410a or other native sequences may also be created. The invention includes transgenic plants, preferably strawberries, including these nucleic acid molecules as well as methods of using these nucleic acid molecules and polypeptides according to the methods described in this application.

Those skilled in the art will recognize that Wcor410a is not the only sequence which may be used to provide increased freezing tolerance activity in plants, as well as the other uses provided in this application. Other sequences that may be used include WCS19 and WCS120.

The genetic code is degenerate so other nucleic acid molecules, which encode a polypeptide identical to WCOR410 sequences in the figures, may also be used. The sequences of the other nucleic acid molecules may also be varied without changing the polypeptide encoded by the sequence. Variants of Wcor410 may occur naturally, for example, by mutation, or may be made, for example, with polypeptide engineering techniques such as site directed mutagenesis, which are well known in the art for substitution of amino acids. For example, a hydrophobic residue, such as glycine can be substituted for another hydrophobic residue such as alanine. An alanine residue may be substituted with a more hydrophobic residue such as leucine, valine or isoleucine. A negatively charged amino acid such as aspartic acid may be substituted for glutamic acid. A positively charged amino acid such as lysine may be substituted for another positively charged amino acid such as arginine. Consequently, the nucleic acid molecule constructs described below and in the accompanying examples for the preferred nucleic acid molecules, vectors, and transformants of the invention are merely illustrative and are not intended to limit the scope of the invention.

The sequences of the invention can be prepared according to numerous techniques. The invention is not limited to any particular preparation means. For example, the nucleic acid molecules can be produced by cDNA cloning, genomic cloning, cDNA synthesis, polymerase chain reaction (PCR), or a combination of these approaches (Current Protocols in Molecular Biology (F. M. Ausbel et al., 1989).). Sequences may be synthesized using well-known methods and equipment, such as automated synthesizers.

Sequence Identity

The invention includes modified nucleic acid molecules with a sequence identity at least about: >17%, >20%, >30%, >40%, >50%, >60%, >70%, >80% or >90% more preferably at least about >95%, >99% or >99.5%, to a Wcor410 sequence shown in the figures. Preferably about 1, 2, 3, 4, 5, 6 to 10, 10 to 25, 26 to 50 or 51 to 100, or 101 to 250 nucleotides are modified. Identity is calculated according to methods known in the art. Sequence identity is most preferably assessed by the algorithm of the BLAST version 2.1 program advanced search. Sequence identity (nucleic acid and protein) is most preferably assessed by the algorithm of BLAST version 2.1 advanced search. BLAST is a series of programs that are available online, for example, at http://www.ncbi.nlm.nih.gov/BLAST. The advanced blast search (http://www.ncbi.nlm.nih.gov/blast/blast.cgi?Jform=1) is set to default parameters. (ie Matrix BLOSUM62; Gap. existence cost 11; Per residue gap cost 1; Lambda ratio 0.85 default).

References to BLAST Searches Include:

Altschul, S. F., Gish, W., Miller, W., Myers, E. W. & Lipman, D. J. (1990) “Basic local alignment search tool.” J. Mol. Biol. 215:403_{—}410.

Gish, W. & States, D. J. (1993) “Identification of protein coding regions by database similarity search.” Nature Genet. 3:266_{—}272.

Madden, T. L., Tatusov, R. L. & Zhang, J. (1996) “Applications of network BLAST server” Meth. Enzymol. 266:131_{—}141.

Altschul, S. F., Madden, T. L., Schaffer, A. A., Zhang, J., Zhang, Z., Miller, W. & Lipman, D. J. (1997) “Gapped BLAST and PSI_BLAST: a new generation of protein database search programs.” Nucleic Acids Res. 25:3389_{—}3402.

Zhang, J. & Madden, T. L. (1997) “PowerBLAST: A new network BLAST application for interactive or automated sequence analysis and annotation.” Genome Res. 7:649_{—}656.

Hybridization

The present invention also includes nucleic acid molecules that hybridize to a Wcor410a sequence shown in the figures, and that encode peptides or polypeptides exhibiting substantially equivalent activity as that of a WCOR410 polypeptide shown in the figures. Such nucleic acid molecules preferably hybridize to all or a portion of a Wcor410 sequenceor its complement under low, moderate (intermediate), or high stringency conditions as defined herein (see Sambrook et al. (most recent edition) Molecular Cloning: A Laboratory Manual, Cold Spring Harbor Laboratory Press, Cold Spring Harbor, N.Y.; Ausubel et al. (eds.), 1995, Current Protocols in Molecular Biology, (John Wiley & Sons, NY)). The portion of the hybridizing nucleic acids is typically at least 15 (e.g. 20, 25, 30 or 50) nucleotides in length. The hybridizing portion of the hybridizing nucleic acid is at least 80% e.g. at least 95% or at least 98% identical to the sequence or a portion or all of a nucleic acid encoding a Wcor410 polypeptide, or its complement. Hybridizing nucleic acids of the type described herein can be used, for example, as a cloning probe, a primer (e.g. a PCR primer) or a diagnostic probe. Hybridization of the oligonucleotide probe to a nucleic acid sample typically is performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g. SSC or SSPE). Then, assuming that 1% mismatching results in a 1 degree Celsius decrease in the Tm, the temperature of the final wash in the hybridization reaction is reduced accordingly (for example, if sequences having greater than 95% identity with the probe are sought, the final wash temperature is decreased by 5 degrees Celsius). In practice, the change in Tm can be between 0.5 degrees Celsius and 1.5 degrees Celsius per 1% mismatch. Low stringency conditions involve hybridizing at about: 1×SSC, 0.1% SDS at 50° C. High stringency conditions are: 0.1×SSC, 0.1% SDS at 65° C. Moderate stringency is about 1× SSC 0.1% SDS at 60 degrees Celsius. The parameters of salt concentration and temperature can be varied to achieve the optimal level of identity between the probe and the target nucleic acid.

Host Cells Including a Cold Tolerance Nucleic Acid Molecule

In a preferred embodiment of the invention, a plant or yeast cell is transformed with a nucleic acid molecule of the invention or a fragment of a nucleic acid molecule and inserted in a vector.

Another embodiment of the invention relates to a method of transforming a host cell with a nucleic acid molecule of the invention or a fragment of a nucleic acid molecule, inserted in a vector. The invention also includes a vector comprising a nucleic acid molecule of the invention. The Wcor410 nucleic acid molecules can be cloned into a variety of vectors by means that are well known in the art. The recombinant nucleic acid molecule may be inserted at a site in the vector created by restriction enzymes. A number of suitable vectors may be used, including cosmids, plasmids, bacteriophage, baculoviruses and viruses. Suitable vectors are capable of reproducing themselves and transforming a host cell. The invention also relates to a method of expressing polypeptides in the host cells. A nucleic acid molecule of the invention may be used to transform virtually any type of plant, including both monocots and dicots. The expression host may be any cell capable of expressing Wcor410 such as a cell selected from the group consisting of a seed (where appropriate), plant cell, bacterium, yeast, fungus, protozoa, algae, animal and animal cell.

The invention relates to a method of producing a polypeptide extract containing cold tolerance polypeptide comprising culturing a host cell or plant, plant part or seed including a nucleic acid encoding a cold tolerance polypeptide from the Wcor410 family under conditions suitable for nucleic acid expression and then obtaining an extract of the host cell or plant, plant part or seed. The invention also relates to the use of a cold tolerance polypeptide extract for the purposes described in this application. This polypeptide extract can be added directly to a cell, cell culture, food or drugs.

Levels of nucleic acid molecule expression may be controlled with nucleic acid molecules or nucleic acid molecule fragments that code for anti-sense RNA inserted in the vectors described above.

Agrobacterium tumefaciens-mediated transformation, particle-bombardment-mediated transformation, direct uptake, microinjection, coprecipitation and electroporation-mediated nucleic acid molecule transfer are useful to transfer a cold tolerance nucleic acid molecule into seeds (where appropriate) or host cells, preferably plant cells, depending upon the plant species. The invention also includes a method for constructing a host cell capable of expressing a nucleic acid molecule of the invention; the method comprising introducing into said host cell a vector of the invention. The genome of the host cell may or may not also include a functional Wcor410 gene. The invention also includes a method for expressing a Wcor410 polypeptide in the host cell or a plant, plant part, seed or plant cell of the invention, the method comprising culturing the host cell under conditions suitable for gene expression. The method preferably also includes recovering the expressed polypeptide from the culture.

The invention includes the host cell comprising the recombinant nucleic acid molecule and vector as well as progeny of the cell. Preferred host cells are fungal cells, yeast cells, bacterial cells, mammalian cells, bird cells, reptile cells, amphibious cells, microorganism cells and plant cells. Host cells may be cultured in conventional nutrient media. The media may be modified as appropriate for inducing promoters, amplifying genes or selecting transformants. The culture conditions, such as temperature, composition and pH will be apparent. After transformation, transformants may be identified on the basis of a selectable phenotype. A selectable phenotype can be conferred by a selectable marker in the vector.

Transgenic Plants and Seeds

Plant cells are useful to produce tissue cultures, seeds or whole plants. The invention includes a plant, plant part, seed, or progeny thereof including a host cell transformed with a wcor410 nucleic acid molecule. The plant part is preferably a leaf, a stem, a flower, a root, a seed or a tuber.

The invention includes a transformed (transgenic) plant having increased cold tolerance, the transformed plant containing a nucleic acid molecule sequence encoding for a cold tolerance polypeptide activity and the nucleic acid molecule sequence having been introduced into the plant by transformation under conditions whereby the transformed plant expresses a cold tolerance protein in active form.

The methods and reagents for producing mature plants from cells are known in the art. The invention includes a method of producing a genetically transformed plant which expresses Wcor410 polypeptide by regenerating a genetically transformed plant from the plant cell, seed or plant part of the invention. The invention also includes the transgenic plant produced according to the method. Alternatively, a plant may be transformed with a vector of the invention.

The invention also includes a method of preparing a plant with increased cold tolerance, the method comprising transforming the plant with a nucleic acid molecule which encodes a Wcor410 polypeptide, or a polypeptide encoding a cold tolerance polypeptide capable of increasing cold tolerance in a cell, and recovering the transformed plant with increased cold tolerance.

The plants (monocots and dicots) whose cells may be transformed with a nucleic acid molecule of this invention and used to produce transgenic plants include, but are not limited to the following:

Target Plants:

Group I (Transformable Preferably Via Agrobacteriurm tumefaciens)

Arabidopsis

Potato

Tomato

Brassica

Cotton

Sunflower

Strawberries

Spinach

Lettuce

Rice

Group II (Transformable Preferably Via Biolistic Particle Delivery Systems (Particle Bombardment)

Soybean

Rice

Corn

Wheat

Rye

Barley

Atriplex

Salicornia

The nucleic acid molecule may also be used with other plants such as oat, barley, hops, sorgum, alfalfa, sunflower, alfalfa, beet, pepper, tobacco, melon, squash, pea, cacao, hemp, coffee plants and grape vines. Trees may also be transformed with the nucleic acid molecule. Such trees include, but are not limited to maple, birch, pine, oak and poplar. Decorative flowering plants such as carnations and roses may also be transformed with the nucleic acid molecule of the invention. Plants bearing nuts such as peanuts may also be transformed with the cold tolerance nucleic acid molecule.

In a preferred embodiment of the invention, plant tissue cells or cultures which demonstrate cold tolerance are selected and plants which are cold tolerant are regenerated from these cultures. Methods of regeneration will be apparent to those skilled in the art These plants may be reproduced, for example by cross pollination with a plant that is cold tolerant or a plant that is not cold tolerant. If the plants are self-pollinated, homozygous cold tolerant progeny may be identified from the seeds of these plants, for example by growing the seeds in a saline environment, using genetic markers or using an assay for cold tolerance. Seeds obtained from the mature plants resulting from these crossings may be planted, grown to sexual maturity and cross-pollinated or self-pollinated.

The nucleic acid molecule is also incorporated in some plant species by breeding methods such as back crossing to create plants homozygous for the cold tolerance nucleic acid molecule.

A plant line homozygous for the cold tolerance nucleic acid molecule may be used as either a male or female parent in a cross with a plant line lacking the cold tolerance nucleic acid molecule to produce a hybrid plant line, which is uniformly heterozygous for the nucleic acid molecule. Crosses between plant lines homozygous for the cold tolerance nucleic acid molecule are used to generate hybrid seed homozygous for the cold tolerance nucleic acid molecule.

### References

1. Hughes, M. A. & Dunn, M. A. The molecular biology of plant acclimation to low temperature. J. Exp. Bot. 47: 291-305 (1996).

2. Thomashow, M. F. Plant Cold acclimation: freezing tolerance genes and regulatory mechanisms. Annu. Rev. Plant Physiol. Plant Mol. Biol. 50: 571-599 (1999).

3. Breton, G., Ouellet, F., Danyluk, J. & Sarhan, F. Biotechnological application of plant freezing associated proteins. Biotechnol. Annu. Rev. 6: 57-99 (2000).

4. Houde, M., Daniel, C., Lachapelle, M., Allard, F., Laliberté, S. & Sarhan, F. Immunolocalization of freezing-tolerance-associated proteins in the cytoplasm and nucleoplasm of wheat crown tissues. Plant J. 8: 583-593 (1995).

5. Limin, A. E., Houde, M., Chauvin, L. P., Fowler D. B. & Sarhan, F. Chromosome mapping of low-temperature induced Wcs120 family genes and regulation of cold-tolerance expression in wheat. Mol. Gen. Genet. 253: 720-727 (1997).

6. Sarhan, F., Ouellet, F. & Vazquez-Tello, A. The wheat Wcs120 gene family: a useful model to understand the molecular genetics of freezing tolerance in cereals. Physiol. Plant. 101: 439-445 (1997).

7. Danyluk, J., Houde, M., Rassart, É. & Sarhan, F. Differential expression of a gene encoding an acidic dehydrin in chilling sensitive and freezing tolerant gramineae species. FEBS Lett. 344: 20-24 (1994).

8. Danyluk, J., Perron, A., Houde, M., Benhamou, N. & Sarhan, F. Accumulation of a major plasma membrane-associated protein during cold acclimation of wheat. Plant Cell 10: 623-638 (1998).

9. Steponkus, P. L., Uemura, M. & Webb, M. S. Membrane destabilization during freeze-induced dehydration. In Plant responses to cellular dehydration during environmental stress, T. J. Close and E. A. Bray, eds (Rockville, Md.: American Society of Plant Physiologists), pp. 37-47 (1993).

10. Mathews, H., Wagoner, W., Kellogg, J. & Bestwick, R. Genetic transformation of strawberry: stable integration of a gene to control biosynthesis of ethylene. In Vitro Cell. Dev. Biol. 31: 36-43 (1995).

11. Kasuga, M., Liu, Q., Miura, S., Yamaguchi-Shinozaki, K. & Shinozaki, K. Improving plant drought, salt, and freezing tolerance by gene transfer of a single stress-inducible transcription factor. Nature Biotechnol. 17: 287-291 (1999).

12. Khanizadeh, S., Buszard, M., Lareau, M. & Bagnara, D. ‘Chambly’ strawberry. Hort. Sci. 25: 984-985 (1990).

13. Murashige, T. & Skoog, F. A. A revised medium for rapid growth and bioassays with tobacco tissue cultures. Physiol. Plant. 15: 473-497 (1962).

14. Gamborg, 0. L., Miller, R. A. & Ovima, K. Nutrient requirement of suspension cultures of soybean root cultures. Exp. Cell Res. 50: 151-158 (1968).

### LEGENDS TO FIGURES

FIG. 1: Transformation and Regeneration of the Strawberry Elite Cultivar Chambly.

The freezing tolerance-associated gene Wcor410a⁸ cloned in the sense orientation was inserted in the expression vector pBI121 (Clontech) by replacing the gus gene. The recombinant vector pBI121-Wcor410a was introduced into Agrobacterium tumefaciens strain GV3101. Three to four weeks old shoot cultures of the strawberry cultivar Chambly (Fragaria×anassassa cv Chambly¹²) were maintained in vitro on the propagation medium MS containing 1 mg/L benzylaminopurine (BA), 0.1 mg/L indolbutyric acid (IBA), 0.01 mg/L gibberellic acid (GA3), 30 g/L sucrose, pH 5.8 and 0.7% agar. The cultured shoots (5 to 10 mm), were cut into 2-3 mm explants that were incubated in A. Tumefaciens suspensions for 90 minutes in the liquid induction medium (MS basal salts¹³ containing B5 vitamins¹⁴, 3% sucrose supplemented with 2 mg/L thidiazuron, and 50 μM acetosyringone, at pH 5.8). The explants were then blotted on a sterile filter paper and cultured with the abaxial surface in contact with the solid induction medium. After two days of cocultivation, the explants were transferred for 4 weeks to the same medium supplemented with 500 mg/L carbenicellin, 500 mg/L of cefotaxime, and 50 mg/L kanamycin. The first subculture was transferred for another 8 weeks onto the same medium containing 1 mg/L thidiazuron. The kanamycin selection pressure was then increased by steps of 100 mg/L at intervals of 4-6 weeks up to a kanamycin concentration of 450 mg/L in the propagation medium. For shoot induction and regeneration, the cultures were incubated at 24° C. with a 16 hour photoperiod (50 μmol m−² s⁻¹) on the propagation medium. Rooting was promoted in the same medium at half strength containing 75 mg/L of kanamycin without hormones.

A) Selection of transgenic explants on 450 mg/L kanamycin.

B) Transgenic shoot regeneration.

C) Regenerated transgenic strawberry plant.

FIG. 2A: Protein Extraction and WCOR410 Quantitation in the Different Strawberry Lines.

Antibodies directed against the WCOR410 protein were used to determine the level of expression in the regenerated transgenic strawberry plants and to select the lines with the strongest expression. Total proteins were extracted from frozen plant tissues as previously described⁸. Proteins, solubilized in sample buffer (60 mM Tris-HC1, pH 6.8, 10% w/v glycerol and 2% w/v SDS), were quantitated using the Bio-Rad Dc Protein Assay. Equal amounts of protein were separated on a 12% SDS-PAGE gel (a load control stained with Coomassie Blue R-250 is shown under the immunoblot) and transferred electrophoretically for 1 hour to a nitrocellulose membrane (HYBOND-C, Amersham) without SDS in the transfer buffer. The membranes were blocked in a 4% (w/v) solution of reconstituted skimmed milk powder prepared in PBS containing 0.2% (v/v) Tween-20, and then probed with the anti-WCOR410 antibody at a 1:10000 dilution for 1h. After washing with PBS-Tween, the proteins recognized by the primary antibody were revealed with a horseradish peroxydase-coupled anti-rabbit IgG (Jackson Immunoresearch Inc.) at a 1:20000 dilution. The complexes were visualized using the ECL chemiluminescent detection system (Amersham) and X-OMAT-RP film (Eastrnan-Kodak, Rochester, N.Y.). Wheat: 2 weeks cold-acclimated winter wheat T aestivum cv. Norstar; WT: wild type Fragaria×anassassa cv. Chambly; 30, 24 and 8A represent three independent non-acclimated transgenic lines.

FIG. 2B: WCOR410 Quantitation in Different Tissues of Transgenic Line L8A by Immunoblot Analysis.

To estimate the relative abundance of WCOR410 proteins in various tissues, the same amount of protein (quantitated with the Bio-Rad Dc Protein Assay) were separated on a SDS-PAGE gel along with total leaf proteins from the cold-acclimated cultivar Fredrick. The proteins were transferred to a nitrocellulose membrane and immunoblot analysis was performed as described above. The relative levels of WCOR410 proteins were then determined by densitometry (Molecular Dynamics personal densitometer and the ImageQuaNT 4.2 software) using the level of cold-acclimated wheat WCOR410 protein as reference.

FIG. 2C: Determination of Freezing Tolerance and Membrane Stability.

Freezing tolerance of transgenic and wild type strawberry was assessed by the electrolyte leakage method. Intact leaves from each line were washed in distilled water, blotted on filter paper, placed in a petri dish and covered with humid sand. The samples were placed in a programmable freezer at −2° C. for 3 hours for temperature equilibration and ice formation. At the end of this period, the sand was completely frozen. The temperature was then lowered at a rate of 1° C./hour. Samples were removed at temperatures from −6 to −19° C. After freezing, leaves were allowed to thaw overnight at 4° C. The thawed leaves were incubated in test tubes containing 25 ml of distilled water and shaken at room temperature overnight. The amount of electrolytes released by the tissues was measured with a conductivity meter (VWR Scientific). The relative conductivities of the supernatant from leaves that had been soaked without any freezing or heat treatments and those that were heated at 100° C. for 30 minutes were taken as 0% and 100%, respectively. The level of freezing tolerance of a particular strawberry tissue was expressed as the temperatures which caused 50% electrolyte leakage (LT₅₀).

FIG. 3: Effect of Freezing on Excised Leaves.

Intact leaves were frozen at −15° C. as described in FIG. 2B. Wild type leaves (top row) are clearly dead and discoloured after the freezing test while transgenic leaves (L30, L24, and L8A from left to right) appear to be intact and remain healthy and green (bottom row).

FIG. 4: Effect of Freezing on Whole Plants

Three weeks cold-acclimated plants were uprooted and frozen at −15° C. as described in FIG. 2C. A. The wild type plant is clearly dead and discoloured after the freezing test while in B, the transgenic plant (line L8A) appears to be intact and remain green.

The present invention has been described in detail and with particular reference to the preferred embodiments; however, it will be understood by one having ordinary skill in the art that changes can be made thereto without departing from the spirit and scope of the invention.

All articles, patents and other documents described in this application (including Genbank sequences and/or accession numbers) are incorporated by reference in their entirety to the same extent as if each individual publication, patent or document was specifically and individually indicated to be incorporated by reference in its entirety. They are also incorporated to the extent that they supplement, explain, provide a background for, or teach methodology, techniques and/or compositions employed herein.

1. A method of increasing the viability of a plant cell comprising transforming a plant with a nucleic acid encoding a cold tolerance polypeptide from the Wcor410 family and combining this treatment with a period of low temperature exposure.

2. A method of improving low temperature tolerance comprising transforming a plant with a nucleic acid encoding a cold tolerance polypeptide from the Wcor410 family and combining this treatment with a period of low temperature exposure.

3. A method of improving freezing tolerance of a plant comprising transforming a plant with a nucleic acid encoding a cold tolerance polypeptide from the Wcor410 family and combining this treatment with a period of low temperature exposure.

4. A method of improving cross-stress tolerance comprising transforming a plant with a nucleic acid encoding a cold tolerance polypeptide from the Wcor410 family and combining this treatment with a period of low temperature exposure.

5. A method of improving membrane stability comprising transforming a plant with a nucleic acid encoding a cold tolerance polypeptide from the Wcor410 family.

6. A method of increasing the viability of a plant cell comprising transforming a plant with a nucleic acid encoding a cold tolerance polypeptide from the Wcor410 family.

7. A method of improving low temperature tolerance comprising transforming a plant with a nucleic acid encoding a cold tolerance polypeptide from the Wcor410 family.

8. A method of improving freezing tolerance of a plant comprising transforming a plant with a nucleic acid encoding a cold tolerance polypeptide from the Wcor410 family.

9. A method of improving fruit and leafy vegetable stability at low temperature comprising transforming a plant with a nucleic acid encoding a cold tolerance polypeptide from the Wcor410 family.

10. A method of improving seed stability and shelf life at low temperature comprising transforming a plant with a nucleic acid encoding a cold tolerance polypeptide from the Wcor410 family.

11. A method of prolonging shelf life of fruit and leafy vegetable at low temperature comprising transforming a plant with a nucleic acid encoding a cold tolerance polypeptide from the Wcor410 family.

12. A method of improving the metabolic stability and activity of a cell stored at low temperature comprising transforming the cell with a nucleic acid encoding a cold tolerance polypeptide from the Wcor410 family.

13. A method of cryoprotection of a cell for biomedical applications comprising transforming a cell with a nucleic acid encoding a cold tolerance polypeptide from the Wcor410 family.

14. A method of improving and increasing the shelf life and quality of frozen food comprising adding to the food an effective amount of a cold tolerance polypeptide from the Wcor410 family.

15. A method of improving and increasing the shelf life of a drug stored at low temperature comprising adding to the drug an effective amount of a cold tolerance polypeptide from the Wcor410 family.

16. A method of improving plant productivity comprising transforming a plant with a nucleic acid encoding a cold tolerance polypeptide from the Wcor410 family.

17. A method of improving plant resistance to osmotic stress comprising transforming a plant with a nucleic acid encoding a cold tolerance polypeptide from the Wcor410 family.

18. A method of improving plant resistance to pathogen stress comprising transforming a plant with a nucleic acid encoding a cold tolerance polypeptide from the Wcor410 family.

19. A method of transforming a plant with a nucleic acid comprising contacting plant with a Agrobacterium tumefaciens suspension in the presence of at least 450 mg/L of kanamycin.

20. The method of any one of claims 1 to 18 wherein the Wcor410 family comprises Wcor410a, Wcor410b and Wcor410c.

21. The method of any one of claims 1 to 19 wherein the nucleic acid comprises the wcor410 nucleic acid in FIG. 1 (SEQ ID NO:1).

22. The method of any one of claims 1 to 19 wherein the nucleic acid comprises nucleotide nos. 87 to 875 of (SEQ ID NO:1).

23. The method of any one of claims 1 to 19 wherein the nucleic acid comprises a nucleic acid having at least 50% sequence identity to (SEQ D NO:1).

24. The method of any one of claims 1 to 19, wherein the nucleic acid comprises a nucleic acid that hybridizes to (SEQ ID NO:1) under stringent hybridization conditions comprising 55-65 degrees Celsius, 5×SSC, 2% SDS; wash: 60-65 degrees Celsius, 0.1×SSC, 0.1% SDS.

25. The method of any one of claims 1 to 18, wherein the polypeptide comprises the WCOR410 amino acid sequence in FIG. 1 (SEQ ID NO:2).

26. The method of any one claims 1 to 18, wherein the polypeptide comprises a polypeptide having at least 50% sequence identity to (SEQ ID NO:2).

27. A method of producing pure stable transgenic strawberry lines using high kanamycin selection pressure.

28. A host cell comprising a nucleic acid encoding a cold tolerance polypeptide from the Wcor410 family, or progeny of the host cell.

29. The host cell of claim 28, selected from the group consisting of a fungal cell, a yeast cell, a bacterial cell, a microorganism cell and a plant cell.

30. A plant, a plant part, a seed, a plant cell or progeny thereof comprising a nucleic acid encoding a cold tolerance polypeptide from the Wcor410 family.

31. The plant part of claim 30, comprising all or part of a leaf, a flower, a stem, a root or a tuber.

32. The plant, plant part, seed or plant cell of claim 30 or 31, wherein the plant comprises a dicot plant.

33. The plant, plant part, seed or plant cell of claim 30 or 31, wherein the plant comprises a monocot plant.
